(19) United States
(12) Patent Application Publication
(10) Pub. No.: US 2020/0009288 A1
(43) Pub. Date: Jan. 9, 2020

(54) ORTHOTIC DEVICE, ORTHOTIC SYSTEM AND METHODS OF MANUFACTURE AND USE THEREOF

(71) Applicants: Charalampos Geremtzes, Edessa (GR); Dimitrios Moustakas, Kremasti Rhodes (GR)

(72) Inventors: Charalampos Geremtzes, Edessa (GR); Dimitrios Moustakas, Kremasti Rhodes (GR)

(21) Appl. No.: 16/459,699

(22) Filed: Jul. 2, 2019

Related U.S. Application Data
(60) Provisional application No. 62/693,971, filed on Jul. 4, 2018.

(51) Int. Cl. A61L 15/14; A61F 5/01; A61L 15/12; A61F 13/04

(52) U.S. Cl. CPC A61L 15/14; A61F 13/046; A61L 15/12; A61F 5/0118

(57) ABSTRACT

An orthotic device for embracing and supporting a body part (e.g., articulation) of a human or animal. The orthotic device is a single sheet of moldable Polylactic Acid (PLA) material configured to maintain the body part in an optimal position for development and treatment. An orthotic system includes the PLA sheet and a protective layer in the form of a single moldable sheet of polyethylene foam (PEF). The PEF sheet is configured for placement between the PLA sheet and the body part.

### RELATED APPLICATION

This application claims the benefit of U.S. Provisional Patent Application No. 62/693,971, filed on Jul. 4, 2018.

### BACKGROUND

### Technical Field

The present disclosure relates to the medical field and, more specifically to an orthotic device in the form of a moldable splint for immobilizing, embracing and supporting a body part, preferably comprising an articulation, of a human or animal. The present disclosure also relates to an orthotic system including the orthotic device and to methods of manufacture and use thereof.

### Background Information

Immobilization devices such as a cast, a splint, a brace (orthosis) and stiffening apparatuses are used to impart a desired position to a supported portion of the body or to immobilize the supported portion relative to other parts of the body. Traditionally, plaster casting materials have been used because they are very low cost. However, plaster casting materials are heavy and cannot be cleaned or easily removed. Recently, plaster casting materials have been replaced by synthetic casting materials which are lighter in weight and can be cleaned but have a rough exterior surface and are still relatively heavy and bulky.

Plaster materials are associated with important disadvantages such as its weight, the development of dust, the occurrence of sharp and hard edges, and the time required for its application. In addition, it is also often necessary that a cast or other immobilization device be removed for medical consultation or exercise by a therapist and then put back on the patient. The plaster material casts cannot be removed intact and put back on the patient.

Braces have been described which are made of a sheet material impregnated or coated with a curable resin. For example, a known orthopedic casting article comprises a flexible sheet material impregnated or coated with two different resins. The article may be in the form of an orthopedic casting tape or a protective pad comprising a fabric backing that is longitudinally impregnated or coated with two different curable resins, preferably water-curable resins. The orthopedic casts are made by providing a curable casting tape; initiating the cure of the casting tape, e.g., by exposing the casting tape to water; and allowing the casting tape to cure to form an orthopedic cast.

Another known orthopedic casting article comprises a curable resin (e.g., a water curable resin) and a filler (e.g., fibrous materials) associated with the resin. Yet another known orthopedic cast comprises a sheet formed of an open-celled foam sheet impregnated with a water curable resin. Upon activation of the resin impregnated foam sheet and molding the same around the body part, an orthopedic splint is formed.

However, a common drawback of the braces comprising a curable resin is that the curing process takes a considerably time, e.g., around 30 minutes or more, before an orthopedic cast functional enough to support the injury is obtained. Moreover, the curing process is irreversible and it is impossible to shape the cured cast or splint in another position. Therefore the cast or splint has to be changed, i.e., a a new cast or splint needs to be applied at different phases of the injury healing process. In addition, to supply the cast or splint a technician needs to wear gloves.

Another problem is that casting or splinting may be very difficult, especially when it is required to build casts having different angles, e.g., a 90° angle between the foot joint and low leg, since at the same time it is necessary to make sure that a good angle is obtained, that the lamination and pressure is optimal, and that contours are followed. Furthermore, a cast or splint like those available in the prior art, wherein the casting material consists of a water-curable resin, and/or currently used products such as synthetic casts and/or P.O.P. ((Plaster of Paris), have a cure (setting) time that can take more than one hour. During all that time the required casting position needs to be maintained, otherwise the casting material can lose the required position and it might be necessary to start all over again. It is very difficult to keep a patient for 30 minutes or more to up to one hour or more to sit still when he/she is in pain, or when he/she is, like most children, scared.

Thermoplastic materials are now being used for forming casts and braces and other immobilization devices. These thermoplastic materials can be produced in extruded sheets which, when brought to a melt point (50° C. to 100° C.), can be molded and manipulated to conform to and shape around a body part, such as a limb, and then allowed to cool to hardness. These materials can also be reheated, brought back to their original shape and then remolded into a different shape. Compared to other casting materials, the thermoplastic materials provide many advantages including simplicity of use and ease of cleaning.

A known thermoplastic apparatus for immobilization or support of a body part of a human or animal is formed from a sheet of thermoplastic moldable material that is substantially rigid at ambient temperatures and pliable at higher temperatures. The apparatus consists of two elements, which are fastened to one another by means of a fastener for fastening. The fastener is directly attached to the thermoplastic material, so that the fastener allows the thermoplastic apparatus to be removed from and put back on the human or animal body part.

However, a problem associated with the above-mentioned braces made of thermoplastic material is that they lack flexibility, and do not allow the injured body part to undergo slight movements, e.g. swelling. In addition, differences in pressure in the brace (orthosis), e.g., due to movements of a patient carrying the brace or due to swelling of the body part, may induce deformations or distortions in the brace configuration and/or create pressure contacts on the body part. In addition, application of the above-mentioned type of braces on impaired limbs, arms or other body parts, involves the adjustment and fastening of the fasteners to a patient, which is a time-consuming process. Another problem associated with this type of braces is that they are relatively heavy.

It has also been suggested to use cork-like material for manufacturing braces and the like. For instance, there is known a splint made of a disposable material containing a cork-like material such as EVA (ethylene vinyl acetate). However, a problem associated with such material is that it is not breathable. This is an important disadvantage, since for improving wound healing and for permitting better transpiration, it is highly recommended to use braces or the like which are capable of some oxygen/air diffusion. It has been shown in the art that braces that are not sufficiently breathable can cause skin irritation, skin maceration, or skin dryness.

Another problem with currently known braces made of softer materials is that the braces may be or become too soft once applied on a body part, and loose sufficient hardness. Such braces may easily bent or form folds or pressure contacts on the body part, which may result in injury or sub-optimal recovery of the injured body part.

In view of the foregoing, there is a need for an orthotic device and system for immobilizing, embracing and supporting a body part, preferably comprising an articulation, of a human or animal, and corresponding methods of manufacture and use thereof which overcome the foregoing drawbacks of the conventional art.

### SUMMARY

In a first aspect, the present disclosure relates to an orthotic device formed of a single sheet of moldable Polylactic Acid (PLA) material for embracing and supporting a body part of a human or animal comprising an articulation and configured to maintain the body part in an optimal position for development and treatment.

According to features of the present disclosure, the PLA sheet is breathable, light-weight, sanitary and moisture-resistant.

In one embodiment, the PLA sheet is formed with a plurality of perforations uniformly distributed over the total surface area of the PLA sheet. Preferably, the perforations are distributed over 75% to 95% of the total surface area of the PLA sheet.

In another embodiment, the PLA sheet has a thickness in the range of about 1.5 mm to about 5.0 mm, and more preferably in the range of about 1.5 mm to about 3.5 mm, and even more preferably in the range of about 1.8 mm to about 2.0 mm.

In other embodiments, the PLA sheet is breathable, light-weight, waterproof and recyclable.

In yet another embodiment, the PLA sheet is formed with at least one aperture for passage therethrough of a body part of the human or animal.

According to a feature of the orthotic device, the PLA sheet is pre-cut to a preselected configuration suitable for forming to shape for a custom application.

In another aspect, the present disclosure is directed to an orthotic system including the orthotic device according to any of the foregoing aspect and embodiments. In one embodiment, the orthotic system further includes an inner protective layer applied directly onto the body part, and the orthotic device is applied directly onto the inner protective layer. In one exemplary embodiment, the inner protective layer comprises a bandage. In another exemplary embodiment, the inner protective layer comprises a single moldable sheet of polyethylene foam. The orthotic system may further include a fastener for releasably securing the orthotic device onto the body part.

In yet another aspect, the present disclosure is directed to a method of forming a splint using the orthotic device according to the foregoing aspects and embodiments.

Still another aspect of the present disclosure relates to methods for manufacturing the orthotic device according to the foregoing aspects and embodiments.

### BRIEF DESCRIPTION OF THE DRAWINGS

The following detailed description of the embodiments of the disclosure will be better understood when read in conjunction with the accompanying drawings. For the purpose of illustrating the disclosure, there is shown in the drawings embodiments which are presently preferred. It should be understood, however, that the disclosure is not limited to the precise arrangement and instrumentalities shown.

FIG. 1 is a front view of an orthotic device in the form of a moldable splint according to an embodiment of the present disclosure which is suitable for being applied in support of a wrist articulation.

FIG. 2 is a diagrammatic view showing a heating procedure for the moldable splint of FIG. 1 using hot water.

FIG. 3 is a diagrammatic view showing a heating procedure for the moldable splint of FIG. 1 using hot air.

FIG. 4 is a perspective side view showing the application of an inner protective layer in the form of a bandage on an injured part (e.g., the wrist and forearm) of a patient's body in preparation for application of the moldable splint of FIG. 1, according to an embodiment of the present disclosure.

FIG. 5 is a perspective view of the moldable splint of FIG. 1 being applied on a patient's body part after application of the inner protective layer.

FIG. 6 is a side view of the moldable splint of FIG. 1 mounted onto a wrist and undergoing a heating procedure for molding and shaping about the patient's wrist.

FIG. 7 is a perspective view showing the removal of the applied inner protective layer after forming the moldable splint of FIG. 1 onto the patient's wrist.

FIG. 8 is a perspective view of another embodiment of the use of the moldable splint of FIG. 1 similar to FIG. 7, except that the inner protective layer is not applied and the moldable splint is formed directly on the patient's wrist and forearm.

FIG. 9 is a view similar to FIG. 7 and illustrates another embodiment in which the inner protective layer remains in place and is not removed after formation of the moldable splint.

FIG. 10A is a perspective view showing the application of a fastener of a first type over the formed moldable splint shown in FIG. 8 to assist in retaining and secure the moldable splint in place over the patient's injured part.

FIG. 10B is a view similar to FIG. 10A showing the fastener completely applied over the moldable splint.

FIG. 11 is a perspective view showing the application of the fastener of the first type over the formed moldable splint shown in FIG. 9 to assist in retaining and securing the moldable splint in place over the patient's injured part.

FIG. 12A is a perspective view showing the application of a fastener of a second type over the formed moldable splint shown in FIG. 8 to assist in retaining and secure the moldable splint in place over the patient's injured part.

FIG. 12B is a view similar to FIG. 12A showing the fastener completely applied over the moldable splint.

FIG. 13 is a perspective view showing the application of the fastener of the second type over the formed moldable splint shown in FIG. 9 to assist in retaining and secure the moldable splint in place over the patient's injured part.

FIG. 14 is a side view of the moldable splint formed on the patient's injured part shown in FIG. 8 and illustrating another embodiment in which the fastener is not applied over the formed moldable splint in its final application on the patient's injured part.

FIG. 15 is a side view of the moldable splint formed on the patient's injured part shown in FIG. 9 and illustrating another embodiment in which the fastener is not applied over the formed moldable splint in its final application on the patient's injured part.

FIG. 16 is a front view of an orthotic device in the form of a moldable splint according to another embodiment of the present disclosure which is suitable for being applied in support of a wrist articulation.

FIG. 17 is a front view of an orthotic device in the form of a moldable splint according to another embodiment of the present disclosure which is suitable for being applied in support of a wrist articulation.

FIG. 18 is a front view of an orthotic device in the form of a moldable splint according to another embodiment of the present disclosure which is suitable for being applied in support of an ankle articulation.

FIG. 19 is a front view of an orthotic device in the form of a moldable splint according to another embodiment of the present disclosure which is suitable for being applied in support of an elbow articulation.

FIG. 20 is a flowchart illustrating steps for manufacturing a moldable splint according to an embodiment of the present disclosure.

FIG. 21 is a flowchart illustrating steps for manufacturing a moldable splint according to another embodiment of the present disclosure.

FIG. 22 is a flowchart illustrating steps for manufacturing a moldable splint according to another embodiment of the present disclosure.

FIG. 23 is a flowchart illustrating steps for applying a moldable splint to a patient according to an embodiment of the present disclosure.

FIG. 24 is a front view of an inner protective layer for the orthotic system according to another embodiment of the present disclosure.

FIG. 25 is a perspective view of the inner protective layer of FIG. 24 being applied on a patient's body part prior to application of the moldable splint.

FIG. 26 is a perspective view showing the moldable splint applied on the patient's body part directly over the inner protective layer of FIGS. 24 and 25.

FIG. 27 is a perspective view showing the application of a fastener in the form of an elastic band to assist in retaining and securing the moldable splint in place over the patient's body part.

FIG. 28 is a perspective view similar to FIG. 27, but with a front portion of the moldable splint being cut off to better illustrate the applied inner protective layer.

FIG. 29 is a cross-sectional view taken along line 29-29 in FIG. 28.

FIG. 30 is a flowchart illustrating steps for applying a moldable splint to a patient in the embodiment of the orthotic system shown in FIGS. 24-29.

### DETAILED DESCRIPTION

The present disclosure now will be described more fully hereinafter with reference to the accompanying drawings, in which exemplary embodiments of the disclosure are shown. This disclosure may, however, be embodied in many different forms and should not be construed as limited to the embodiments set forth herein; rather, these embodiments are provided so that this disclosure will be thorough and complete, and will fully convey the scope of the disclosure to those skilled in the art.

It should also be understood that the terms “about,” “approximately,” “generally,” “substantially” and like terms, used herein when referring to a dimension or characteristic of a component of the present disclosure, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

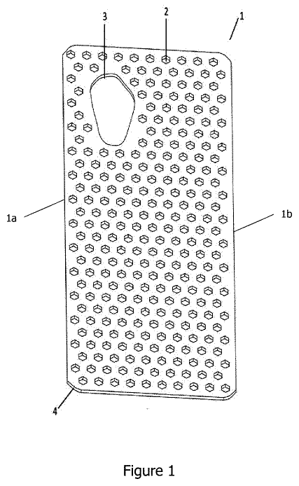

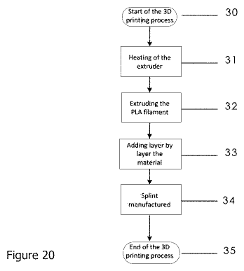

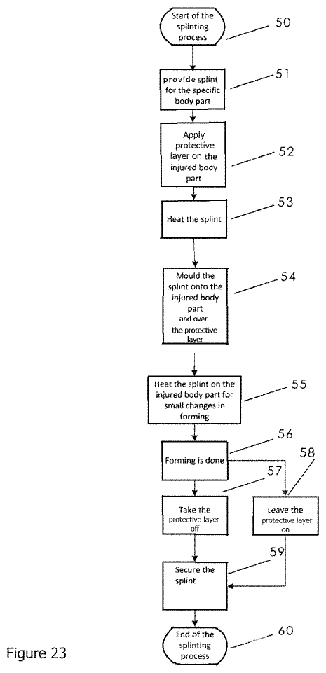

FIGS. 1-19 and 24-29 illustrate various embodiments of orthotic devices and systems according to present disclosure, where similar references numerals designate similar elements and features throughout. FIGS. 20-22 are flowcharts illustrating various embodiments of methods for manufacturing the orthotic devices according to the present disclosure. FIGS. 23 and 30 illustrate embodiments of splinting processes using respective orthoses according to the present disclosure.

FIG. 1 is a front view of an orthotic device or orthosis, generally designated at 1, according to an embodiment of a first aspect of the present disclosure. Orthosis 1 is suitable for embracing and supporting a body part of a human or animal comprising an articulation. It is generally prescribed by a physician to provide correction, support, or protection to the body part. Orthosis 1 is prefabricated without a specific patient in mind (i.e., they can fit any patient). It requires the expertise of a therapist to customize the orthosis to a specific patient, including trimming, molding and bending, with or without heat. Orthosis 1 can be molded in the unique skeletal characteristics of a patient's fractured body part (e.g., wrist, arm, leg, shoulder, head/neck).

In the embodiment shown in FIG. 1, orthosis 1 is in the form of a moldable thermoplastic splint which is suitable for being applied in support of a wrist articulation. It is formed of a single, flat sheet of material which is pre-cut to a preferred configuration that may be later formed to shape for a custom application. Orthosis 1 is light-weight, waterproof, and recyclable, and is designed to maintain the body part in an optimal position for development and treatment.

According to a feature of the present disclosure, orthosis 1 is made of a thermoplastic material which is moldable (e.g., above 50° C.) and breathable. By this construction, when orthosis 1 is heated above a specific temperature it becomes flexible and can be adjusted to the unique skeletal characteristics of the body part to be treated. After orthosis 1 cools down, it becomes rigid again.

The term “flexible” as used herein, refers to a material which is able to adjust readily to different conditions and in particular refers to a material which is able to easily flex and/or bend without breaking.

As used herein, the term “breathable” refers to a material, which allows air to pass to some degree. Such materials keep out water, but also release perspiration/transpiration vapor. This term refers to materials that have been made breathable by means of human mediation, for instance by means of perforation. In this regards, in the present embodiment perforations 2 are provided in the sheet of thermoplastic material for breathability of orthosis 1 once in place on the patient, that is, perforations 2 permit ventilation of the patient's skin when orthosis 1 is in place upon the body part of the patient. Perforations 2 may also facilitate molding of the sheet when shaping the sheet into a splint on the patient. Preferably, between about 75% to about 95% of the total surface area of orthosis 1 is perforated, and for instance about 75%, about 80%, about 90% or about 95% of the total surface area of orthosis 1 is perforated. Perforations 2 are uniformly distributed over the total surface area of orthosis 1. As shown in FIG. 1, perforations 2 are generally uniform and have a diameter generally in the range of about 3 mm to about 10 mm. The diameter of perforation 2 may be greater than the diameter values in this range based on an increase in the thickness of orthosis 1. The amount of perforation provides adequate strength to orthosis 1 while allowing for good air circulation, which will improve skin ventilation and wound healing as compared to non-perforated and/or non-breathable orthotic devices. Problems of skin irritation or skin maceration can advantageously be diminished by means of perforations 2. In alternative embodiments, orthosis 1 may be provided without perforations 2, or perforations 2 may be provided only in certain sections of orthosis 1.

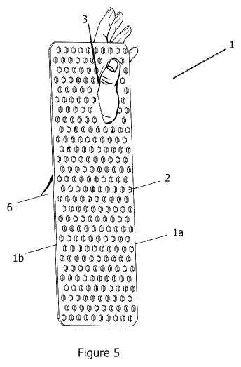

Another feature according to the present disclosure is that the orthosis may also be perforated to provide apertures for body parts, such as thumbs, toes, etc. in the embodiment of FIG. 1, a single aperture 3 is provided for the patient's thumb when orthosis 1 is mounted onto the patient's wrist, as shown for example in FIG. 5.

According to another feature of the present disclosure, the thickness of orthosis 1 designated by reference numeral 4 in FIG. 1, is preferably in the range of about 1.5 mm to about 5.0 mm, more preferably in the range of about 1.5 mm to about 3.5 mm, and even more preferably in the range of about 1.8 mm to about 2.0 mm. These preferred thicknesses allow orthosis 1 to be lightweight without compromising the rigidity necessary for the particular use. For example, when the orthosis according to the present disclosure is used for support of the hand, such as orthosis 1 mounted onto the wrist, a thinner orthosis is preferable, and when the orthosis is used as an immobilization orthosis for the lower extremities, a thicker orthosis is preferred. A variety of thicknesses for the orthosis can be formed into sheets in which the appropriate thickness is chosen for the desired support of the pre-selected body part. Selecting a sheet of the appropriate thickness will eliminate excess bulk in the weight of the orthosis.

As another feature of the orthosis according to the embodiment of the present disclosure, the thermoplastic sheet of material is formed of Polylactic Acid (PLA). While PLA is a thermoplastic material that allows for flexibility and adjustability, the use of PLA allows the orthosis according to the present disclosure to provide sufficient rigidity to the injured body part. The inventors of the present disclosure have discovered that PLA imparts a rigidity on the orthosis of the present disclosure that cannot be achieved by other types of thermoplastic materials, such as Polycaprolactone (PCL), particularly when the orthosis 1 has a thickness within the ranges described above. The flexible and adjustable nature of the PLA orthosis according to the present disclosure allows the injured body part to which the orthosis is mounted to undergo slight movements, e.g., swelling. The flexibility of the PLA material makes the orthosis of the present disclosure suitably deformable in order to conform to contours and the physical reactions of the treated body part (e.g. swelling/de-swelling) while not compromising the rigidity and hardness of the orthosis. Another advantage of using PLA for the orthosis according to the present disclosure is that PLA is a biodegradable, biocompatible, non-toxic and eco-friendly polymer.

Mechanical and material properties of PLA for the orthosis according to the present disclosure are provided below:

Mechanical Properties of MA

| | | |
|---|---|---|
| Elastic (Young's Tensile) Modulus | 3.5 GPa | (0.51 × 10⁶ psi) |
| Flexural Modulus | 4.0 GPa | (0.58 × 10⁶ psi) |
| Shear Modulus | 2.4 GPa | (0.35 × 10⁶ psi) |
| Elongation at Break | 6.0% | |
| Flexural Strength | 80 MPa | (12 × 10³ psi) |
| Tensile Strength (Ultimate UTS) | 50 MPa | (7.3 × 10³ psi) |

Material Properties of MA

| | | |
|---|---|---|
| Density | 1.3 g/cm³ | (78 lb/ft³) |
| Heat Deflection Temperature | 65° C. | (150° F.) |
| at 455 kPa (66 psi) | | |
| Specific Heat Capacity | 1800 J/Kg-K | (0.43 BTU/lb-° F.) |
| Glass Transition Temperature | 60° C. | (140° F.) |
| Melting Onset (Solidus) | 160° C. | (320° F.) |
| Thermal Conductivity | 0.13 W/m-K | (0.075 BTU/h-ft-° F.) |

As a production process, there are two methods for manufacturing polylactic acid (PLA) from lactic acid: the first method uses the cyclic lactic acid dimer called lactide as an intermediate stage; the second method is direct polymerization of lactic acid. The method using the lactide intermediary yields polylactic acid (PLA) with greater molecular weight. This production process is described in the publication Fibre Chemistry Vol, 41, Nov. 6, 2009, by Ozan Avinc and Akbar Khoddami, which is incorporated herein by reference in its entirety.

Suitable PLA's for the orthosis according to the present disclosure include the following commercially available products:
eSun 3D Printer Filament, PLA, 3 mm
eSUN 1.75 mm Red PLA PRO (PLA+) 3D Printer Filament
3D Prima PrimaValue PLA Filament, 1.75 mm
Prima Filaments PS-PLAPRO-175-0750-RD PrimaSelect PLA PRO, 1.75 mm

As noted above, the thickness of orthosis 1 according to the present disclosure allows orthosis 1 to be lightweight but also as rigid as required. Advantageously, the orthosis according to the present disclosure is considerably lighter than prior art orthotic devices. For example, orthosis 1 according to the present disclosure preferably has a weight higher than 60 grams but preferably less than 180 grams.

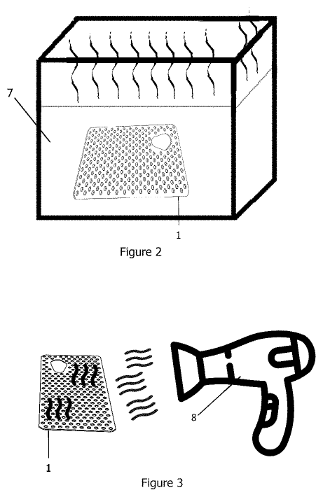

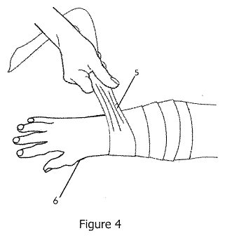

Orthosis 1 according to the present disclosure, which is preferably made of a single flat sheet of pre-cut PLA material, may be shaped onto the patient's body part (i.e., the wrist) by applying heat using a heat source. In one embodiment, the heat source may be a heated water container 7 as shown in FIG. 2. In an alternative embodiment, the heat source may be a thermogun 8 as shown in FIG. 3. Without departing from the spirit and scope of the present disclosure, various other types of heat sources may be used to apply heat orthosis 1 including, but not limited to, a convection oven, a microwave oven and an infrared heater. Orthosis 1 is sufficiently heated to become temporarily elastic so that it can be molded into the shape of the body part to which it is applied. Stated otherwise, orthosis 1 is heated above its glass transition temperature (and below its melting temperature) so as to become thermoformable for shaping within minutes. This temperature should be in the range of about 60° C. to about 90° C. (target temperatures). When made of PLA, orthosis 1 is heated above the PLA glass transition temperature of 60° C. Then orthosis 1 then cools down at room temperature.

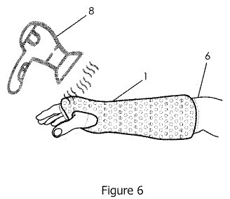

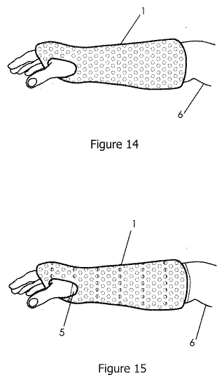

According to another feature of the present disclosure, are inner protective layer is applied directly onto the patient's body part for protecting the body part from the heat applied by the heat source during the forming/molding process. In one embodiment, the inner protective layer is a bandage (e.g., as elastic bandage) that is applied onto the patient's body part in preparation for forming orthosis 1 on the body part. For example, FIG. 4 illustrates the application of a bandage 5 on the patient's body part 6 in preparation for forming orthosis 1 (FIGS. 5-6). In addition to protecting the body part from the heat applied by the heat source during the forming/molding process, the bandage provides further capability to custom fit the orthosis to the particular body part of a particular patient. FIG. 15 shows the completed orthosis 1 which has been applied over the patient's body part 6 with bandage 5 interposed therebetween. Bandage 5 may be made of a suitable fabric material in the appropriate width, thickness and elasticity suitable for the particular purpose and in a length to adequately wrap over the entire portion of the body part on which the orthosis 1 is to be formed. The combination of orthosis 1 and the inner protective layer, such as bandage 5 described above, defines an orthotic system according to another aspect of the present disclosure.

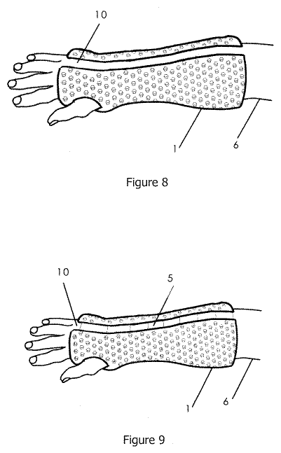

In an alternative embodiment, the inner protective layer (e.g., bandage) is not applied, and orthosis 1 is applied directly onto the patient's body part 6, as for example shown in FIG. 8. FIG. 14 shows the completed orthosis 1 which has been applied directly over the patient's body part 6 without bandage 5.

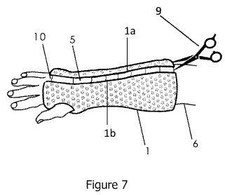

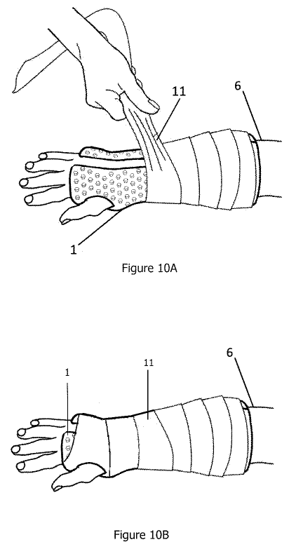

According to an embodiment of the present disclosure, orthosis 1 is initially formed so that it does not completely encircle the patient's body part. As shown in FIG. 7, for example, an overlap slit or opening 10 is formed by overlap longitudinal edges 1a, 1b (FIGS. 1, 5) of orthosis 1 after the initial formation of orthosis 1. This gap or slit 10 allows bandage 5 to be removed, such as by using a cutting device 9, after application of orthosis 1, as for example shown in FIG. 7. In an alternative embodiment, bandage 5 is not removed and remains on the patient's body part after formation of orthosis 1, as for example shown in FIG. 9. FIGS. 14-15 show the completed orthosis 1 one with (FIG. 15) and without (FIG. 14) bandage 5 applied on injured body part 6.

During application of heat, as shown in FIG. 6, for example, orthosis 1 becomes warm, soft and pliable and is formed to the intimate shape of the body part to best stabilize the injury under reduction. Overlap opening 5 formed by overlap edges 1a, 1b of orthosis 1, as shown in FIG. 7, is adjustable in circumference. Thus orthosis 1 is best formed when warm and pliable by applying compressive circumferential force. Once orthosis 1 is cool and rigid in a few moments, this compression can be removed.

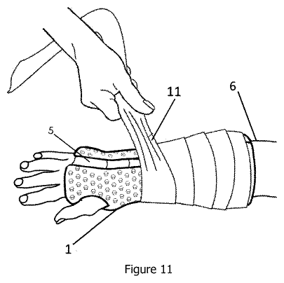

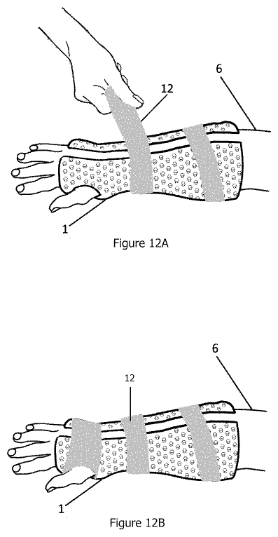

According to the present disclosure, various means are provided for applying the requisite compressive force to orthosis 1. In one embodiment, compression is applied to orthosis 1 by quickly wrapping an elastic band 11, as a fastener of the first type, around the warm and pliable orthosis 1 as soon as it is installed, as for example shown in FIG. 10A (embodiment of orthosis 1 without use of bandage 5) and FIG. 11 (embodiment of orthosis 1 using bandage 5). Elastic band 11 may be made of a fabric or rubberized material in the appropriate width, thickness and elasticity suitable for the particular purpose and in a length to adequately wrap over the entire orthosis 1. Compressive pressure can be varied simply by pulling on band 11 as it is wrapped, as shown in FIG. 10. This process will insure that every part of orthosis 1 is formed to the patient's body part (i.e., the patient's wrist) and voids are not created. Accordingly, elastic band 11 provides further capability to custom fit orthosis 1 to body part 6 of a particular patient. After orthosis 1 is cooled, band 11 is removed as shown for example in FIG. 14 (embodiment of orthosis 1 without use of bandage 5) and FIG. 15 (embodiment of orthosis 1 using bandage 5).

The application of orthosis 1 is completed on injured body part 6 by releasably securing orthosis 1 with a fastener. In one embodiment, one type of fastener may be in form of an elastic band such as described above for elastic band 11. For example, after being used to apply the requisite compressive force to orthosis 1 as described above, elastic band 11 may be completely applied and retained (i.e., is not removed) over orthosis 1 to secure orthosis 1 about body part 6 as shown in FIG. 10B. The combination of orthosis 1 and the fastener (e.g., elastic band 11), without or without the inner protective layer (e.g., bandage 5) as described above, defines an orthotic system according to another aspect of the present disclosure.

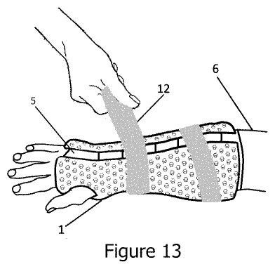

FIGS. 12A, 12B and 13 show another embodiment of a fastener for releasably securing orthosis 1 about body part 6. In this embodiment, the fastener is in the form of a retaining strap 12 as shown in FIG. 12 (embodiment of orthosis 1 without use of bandage 5) and FIG. 13 (embodiment of orthosis using bandage 5). In one embodiment, retaining strap 12 comprises a continuous loop of fabric suitable for hook and loop fastening (e.g., Velcro strap).

It will be appreciated that the use of elastic band 11 or retaining strap 12 as the fastener allows orthosis 1 to be rapidly applied to and removed from injured body part 6 by increasing or decreasing the amount of overlap to more closely fit the injured body part. Alternatively, a plurality of retaining straps or other means for securing orthosis 1 relative to body part 6 are suitable without departing from the spirit and scope of the disclosure.

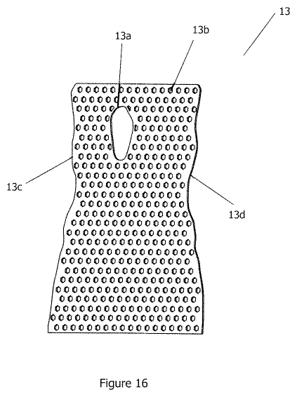

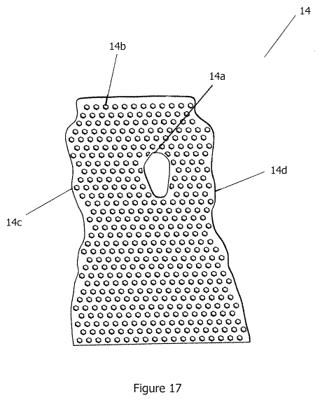

FIGS. 16-19 show alternative embodiments of the orthosis, generally designated with respective reference numerals 13-16, according to the present disclosure for embracing and supporting a body part of a human or animal comprising an articulation.

Orthosis 13 shown in FIG. 16 is suitable for being applied in support of a wrist articulation. Orthosis 13 is formed with a single aperture 3 provided for the patient's thumb, and perforations 13b similar to perforations 2 as described above for orthosis 1 with reference to FIG. 1. Orthosis 13 has opposing longitudinal sides 13c, 13d formed with undulations and/or curved surface portions to facilitate its application over the patient's wrist.

Orthosis 14 shown in FIG. 17 is also suitable for being applied in support of a wrist articulation. Orthosis 14 is formed with a single aperture 14a provided for the patient's thumb, and perforations 14b similar to perforations 2 as described above for orthosis 1 with reference to FIG. 1. Similar to orthosis 13, orthosis 14 has opposing longitudinal sides 14c, 14d formed with undulations and/or curved surface portions as shown in FIG. 17. Orthosis 14 and orthosis 13 are similar to one another, except that they have somewhat different outer peripheral structural configurations as defined by their respective opposing longitudinal sides.

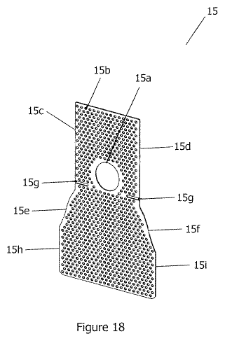

Orthosis 15 shown in FIG. 18 is suitable for being applied in support of an ankle articulation. Orthosis 15 is formed with a single aperture 15a for a body part and perforations 15b similar to perforations 2 as described above for orthosis 1 with reference to FIG. 1. Orthosis 15 has a first part with opposing longitudinal side portions 15c, 15d, a second part with opposing longitudinal side portions 15e, 15f, and a third part with opposing longitudinal side portions 15h, 15i. Opposing open slits 15g are formed at an intersection between the first and second parts of orthosis 15. The purpose/function of slits 15g is for being able to fold the material of orthosis 15 when it is pliable without forming edges and making it too thick. Opposing longitudinal sides 15c, 15d are generally linear and parallel to one another. Opposing longitudinal sides 15h, 15i are generally linear and parallel to one another. Opposing longitudinal sides 15e, 15f are disposed between opposing longitudinal sides 15c, 15d and 15h, 15i and are formed with undulations and/or curved surface portions as shown in FIG. 18. Aperture 15a is formed in the first portion of orthosis 15 proximate the intersection between the first and second portions. Orthosis 15 is symmetrical about a line extending through a center of aperture 15a and parallel to opposing longitudinal sides 15c, 15d and 15e, 15f.

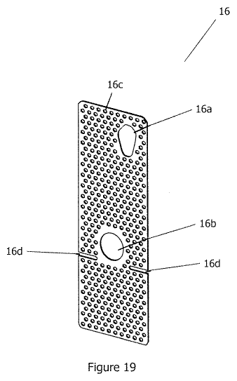

Orthosis 16 shown in FIG. 19 is suitable for being applied in support of an elbow articulation. Orthosis 16 has a first part formed with two apertures 16a, 16b and a second part separated from the first part by opposing open slits 16d similar in structure and function as opposing open slits 15g shown in FIG. 18. The first and second parts of orthosis 16 are formed with perforations 16c similar to perforations 2 as described above for orthosis 1 with reference to FIG. 1. Aperture 16b is provided proximate to and closer to opposing open slits 16d than aperture 16a. The first and second parts of orthosis 16 have different lengths and, in this embodiment, the first part is longer than the second part as shown in FIG. 19.

The structural features and advantages for orthoses 13-16 in FIGS. 16-19 are the same as for orthosis 1 described above with reference to FIGS. 1-14. In addition to the variation in shape and location of aperture 3, orthoses 15 and 16 shown in FIGS. 18 and 19 differ from orthoses 1, 13 and 14 in that orthoses 15 and 16 are further provided with slits 15g, 16d, respectively, whose purpose/function is to facilitate folding of the orthosis material when it is pliable without forming edges and making it to thick as described above. Orthosis 16 shown in FIG. 19 further differs from orthoses 1 and 13-15 in that orthosis 16 is provide with two apertures for body parts instead of one.

FIGS. 20-22 are flowcharts illustrating methods for manufacturing the orthotic device according to various embodiments of the present disclosure.

The flowchart in FIG. 20 illustrates steps for manufacturing a PLA splint according to the present disclosure by a 3D printing process. New developments in 3D printing technologies offer multiple possible new frontiers to improve patient care, satisfaction, and offer potential financial and investment opportunities. In this embodiment, the 3D printing process starts at Step 30. The method includes heating a 3D printer extruder (Step 31), extruding the PLA filament (Step 32), adding the PLA material layer by layer (Step 33) until the splint is manufactured (Step 34). Step 35 in designates the end of the 3D printing process.

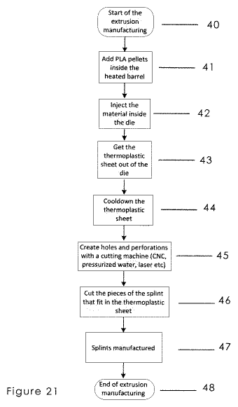

The flowchart in FIG. 21 illustrates steps for manufacturing a PLA splint according to the present disclosure by an extrusion molding process. In this embodiment, the extrusion molding process starts at Step 40. PLA pellets are added inside a heated barrel (Step 41) and the resulting material is injected inside a die to form a thermoplastic sheet (Step 42). The thermoplastic sheet is then removed from the die (Step 43) and is allowed to cool down (Step 44). Thereafter, the corresponding holes and perforations for the splint as described above are formed in the thermoplastic sheet with a cutting machine (e.g., by CNC machining, pressurized water, laser, etc.) (Step 45). The thermoplastic sheet is then subjected to a cutting operation to provide the splints (Step 46), resulting in the manufacture of the splints (Step 47). Step 48 in designates the end of the extrusion molding process.

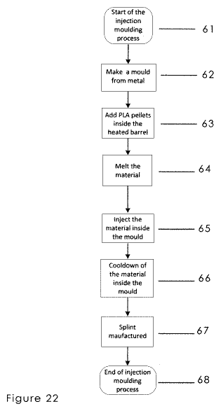

The flowchart in FIG. 22 illustrates steps for manufacturing a PLA splint according to the present disclosure by an injection molding process. In this embodiment, the injection molding process starts at Step 61. A mold is prepared from a metal (Step 62). PLA pellets are added inside a heated barrel (Step 63) and the material is subjected to a melting operation (Step 64). The incited material is then injected inside of the mold (Step 65). The melted material is then allowed to cool inside of the mold (Step 66), resulting in the manufacture of the splint (Step 66). Step 48 in designates the end of the extrusion molding process.

The PLA splints manufactured by any of the foregoing methods according to the present disclosure exhibit the various preferred structural characteristics and advantages described above with reference to FIGS. 1-19.

FIG. 23 is a flowchart illustrating an embodiment of a splinting process using the orthosis (splint) according to the present disclosure. The process will be described with reference to FIGS. 1-15.

At the start of the process (Step 50), orthosis 1 (splint) according to the present disclosure is first provided (Step 51). Orthosis 1 is provided as a kit to the individual, the orthopedic specialist, physician, technician, first responder or other entity. In certain embodiments as described above, the kit further includes an inner protective layer (e.g., elastic bandage 5) and a fastener (e.g., elastic band 11 or retaining strap 12) as describe above which, together with orthosis 1, defines an orthotic system according to the present disclosure. The appropriate kit type and size for the injured body part to be supported is selected. Depending on the kit type selected, the orthosis is applied on the protective layer (FIGS. 6, 7, 9, 11, 13, 15) or directly on the injured body part 6 (FIGS. 8. 10, 12, 14).

In this embodiment, bandage 5 (FIG. 4) as the inner protective layer is then applied to injured body part 6 (wrist) (Step 52). To form orthosis 1, a heat source is then applied to the orthosis to until the orthosis is sufficiently pliable (i.e., to induce elasticity) to allow it to be molded into the shape of the body part to which it is applied (Step 53). Stated otherwise, orthosis 1 is heated above its glass transition temperature (and below its melting temperature) so as to become thermoformable for shaping within minutes. This temperature should be in the range of about 60° C. to about 90° C. (target temperatures). When made of PLA, orthosis 1 is heated above the PLA glass transition temperature of 60° C. The heat source can be hot water (FIG. 2) or a thermogun (FIG. 3). Alternatively, the heat source can be an oven or microwave.

Once orthosis 1 is sufficiently heated and pliable, approximately 5-10 minutes, for example, it is mounted and molded onto injured body part 6 (Step 54; FIGS. 5-6). In this embodiment, orthosis 1 is mounted directly onto bandage 5 (FIG. 9). If bandage 5 is not used, orthosis 1 is mounted directly onto injured body part 6 (FIG. 8). In this state, the heated orthosis 1 is manipulated to form a physiological conforming splint on injured body part 6. Gentle manual pressure may be used to form the splint into the correct shape, taking into account the individual characteristics of the intended user and the purpose for the splinting. During this formation process, orthosis 1 is also applied with compressive circumferential force as described above with reference to FIGS. 10A and 11, for example. Once orthosis 1 is cool and rigid in a few moments, this compression can be removed.

The heated orthosis 1 remains elastic and pliable for several minutes at room temperature, generally long enough for one skilled in the art to form a physiologically conforming splint, but orthosis 1 may be subjected to the heat source at any time to effect additional small changes in the forming/molding of orthosis 1 on injured body part 6 (Step 55) and/or to restore elasticity and prolong working time. The physiological conforming splint is then allowed to cool at room temperature on injured body part 6 to complete the forming process (Step 56).

After formation of orthosis 1 on injured body part 6, bandage 5 is taken off (Step 57). This can be accomplished, for example, by cutting bandage 5 along overlap slit or opening 10 that is formed by overlap edges 1a, 1b of orthosis 1, as described above, and removing cut pieces of bandage 7 via opening 10 and/or aperture 3 (FIG. 7). Alternatively, bandage 5 may be left on injured body part 6 as an element of the orthotic system according to the present disclosure (Step 58; FIG. 9).

When the technician is satisfied with the final shape and fit of the physiologically conforming splint, the formation of orthosis 1 is done (Step 56). Application of orthosis 1 onto injured body part 6 is completed by releasably securing orthosis 1 with a fastener as described above. For example, the fastener may be in the form of a retaining strap 12 as shown in FIG. 12 (embodiment of orthosis 1 without use of bandage 5) or FIG. 13 (embodiment of orthosis using bandage 5). In one embodiment, retaining strap 12 comprises a continuous loop of fabric suitable for hook and loop fastening (e.g., Velcro strap). Alternatively, the fastener may be in form of an elastic band such as described above for elastic band 11. For example, after being used to apply the requisite compressive force to orthosis 1 as described above, elastic band 11 may be completely applied and retained (i.e., is not removed) over orthosis 1 to secure orthosis 1 about body part 6 as shown in FIG. 10B. In other embodiments, that fastener may be in the form of a plurality of retaining straps or other suitable means for securing orthosis 1 onto injured body part 6 without departing from the spirit and scope of the disclosure. It will be appreciated that fastener allows orthosis 1 to be rapidly applied to and removed from injured body part 6 by increasing or decreasing the amount of overlap to more closely fit the injured body part.

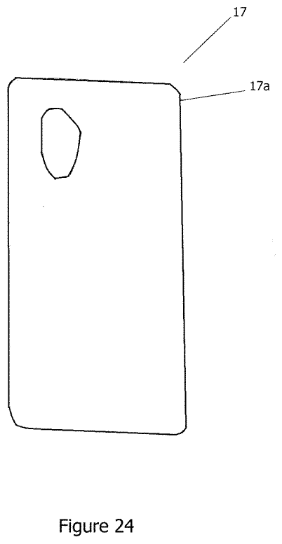

FIGS. 24-30 shows another embodiment of an orthotic system according to the present disclosure. In this embodiment, the orthotic system includes an orthotic device according to any of the embodiments described above with reference to FIGS. 1-23 and an inner protective layer formed of a single moldable sheet of polyethylene foam (PEF).

In the present embodiment, the PEF sheet is formed of a single, flat sheet of material which is pre-cut to a preferred configuration that may be later formed to shape for a custom application together with a corresponding orthosis according to the present disclosure. As the inner protective layer, the PEF sheet is used in this embodiment in place of the bandage as described above for the previous embodiments.

PEF is a durable (wear and tear resistant), semi-rigid and lightweight closed-cell material. As compared to open-cell foams, PEF exhibits strength, rigidity, and resistance to water and moisture. PEF is also resistant to solvents and petroleum products (including chemicals and grease), and its antimicrobial property inhibits the growth of mold, mildew, and bacteria (i.e., is impervious to mildew, rot and bacteria). As a resilient material, PEF returns to form after compression, while still yielding enough to provide cushion and security where it is needed. In addition to the foregoing, the inventors of the present disclosure have discovered that PEF is particularly suitable for orthopedics due to the following additional characteristics and properties:

Flexibility

Cushioning

Uniform cell structure

Dimensional stability

Tear/puncture-resistant

Temperature resistant between 60° C. and 90° C.

Thermally insulative

Naturally resistant to low fire that can be improved by a suitable formulation

Non-abrasive

Non-dusting

Odorless

Cost/labor efficient (cost effective and easy to fabricate)

Excellent in shock absorption & vibration dampening properties

CFC (Chlorofluorocarbons) free

Ozone friendly

Recyclability

Example properties for PEF are provided below:

| | | | |
|---|---|---|---|
| Density | kg/m³ | 25 to | 185 |
| Thermal conductivity | W/m · K | 0.034 to | 0.067 |
| 10% compression stress | MPa | 0.012 to | 0.160 |
| 50% compression stress | MPa | 0.080 to | 0.33 |
| Compression set, 22 h, 25% | % | 2 to | 13 |
| Tensile strength | MPa | 0.140 to | 3.9 |
| Elongation at break | % | 80 to | 425 |
| Water absorption, 7 days | % | &lt; 1 to | &lt; 2.5 |
| Service temperatures | ° C. | −80 to | 100 |

FIG. 24 is a front view of a PEF sheet, generally designated with numeral 17, as the inner protective layer for the orthotic system according to this embodiment of the present disclosure. PEF sheet 17 is configured for use with orthosis 1 described above with reference to FIG. 1. In this regard, PEF sheet 17 has substantially the same structural configuration (geometry) as orthosis 1, except that PEF sheet 17 is not provided with apertures 2. In an alternative embodiment, PEF sheet 17 may be provided with apertures 2. The thickness of PEF sheet 17 is preferably in the range of about 1.5 mm to about 5.0 mm, and more preferably about 3 mm.

PEF sheet 17 may be adhered to orthosis 1 using a suitable adhesive. For example, a surface of PEF sheet 17 on which orthosis 1 is applied may be coated with an adhesive an adhesive on which a peelable backing sheet is applied. During use, after PEF sheet 17 is applied on the patient's body part, the backing sheet is peeled off and orthosis 1 is attached to the surface of PEF sheet coated with the adhesive. Alternatively, orthosis 1 may be applied directly over PEF sheet 17 without using an adhesive.

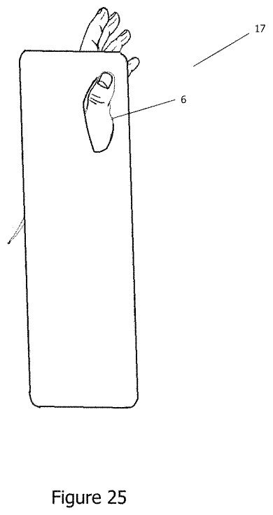

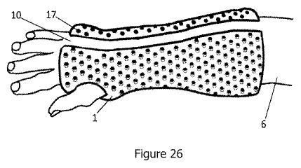

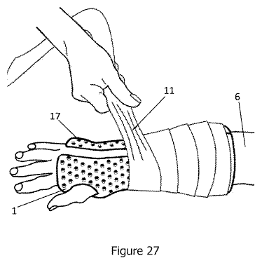

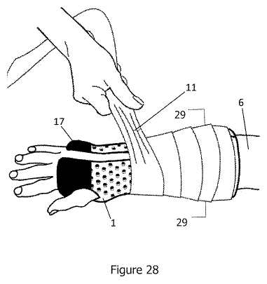

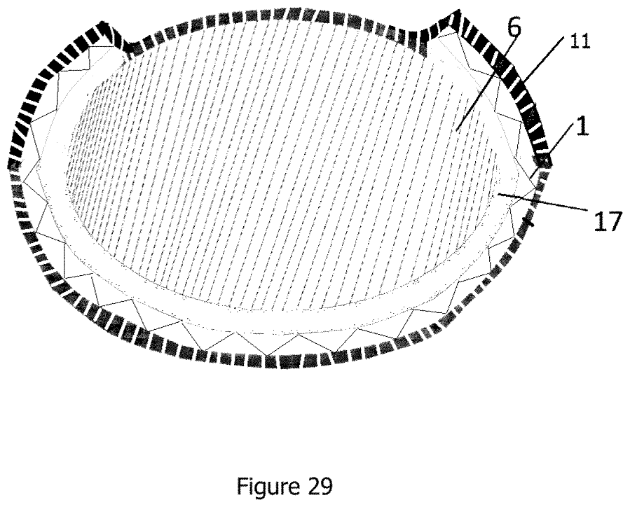

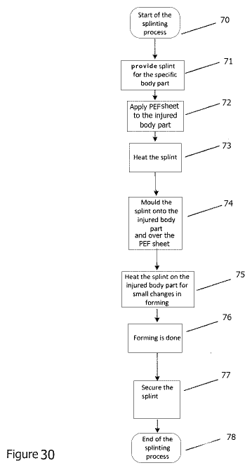

FIG. 25 shows PEF sheet 17 in the process of being applied on a patient's body part 6 prior to application of orthosis 1. FIG. 26 shows orthosis 1 applied on body part 6 directly over PEF sheet 17. FIG. 27 shows the use of elastic band 11 to apply compression to orthosis 1 and/or as a fastener to assist in retaining and securing orthosis 1 in place to body part 6, as described above for the embodiment of FIG. 11. FIG. 28 is view similar to FIG. 27, but with a front portion of orthosis 1 removed to better illustrate PEF sheet 17 applied on the body part. FIG. 29 is a cross-sectional view taken along line 29-29 in FIG. 28.

It will be appreciated that retaining strap 12 as described above for the embodiment of FIG. 13, or other suitable fastener, may be used in place of elastic band 11 to assist in retaining and securing orthosis 1 to body part 6. PEF sheet 17 may have a configuration other than as shown in FIG. 24. For example, PEF sheet 17 may have a configuration similar to any one of orthosis 13, 14, 15 or 16 shown in FIGS. 16-19, respectively, when used in combination with any one of these orthoses to provide the orthotic system. The formation of the orthosis, including application of heat, is as described above with reference to the embodiment shown in FIGS. 1-3.

According to a feature of the present disclosure, PEF sheet 17 effectively protects the body part from the heat during the forming/molding process. In addition to protecting the body part from the heat applied by the heat source during the forming/molding process, PEF sheet 17 provides further capability to custom fit the orthosis to the particular body part of a particular patient. The inventors of the present disclosure have discovered that due to the thermal insulation property of PEF sheet 17, the splint (e.g., PLA sheet) remains moldable for more than 1 minute, providing sufficient time for medical professionals to mold the splint onto the patient's injured body part. Because of its rest properties, the resulting orthosis is waterproof and lightweight, as well as resistant to odors, bacteria, mildew and rot and with increased sock absorption and vibration dampening properties, among other properties and characteristic described above for PEF sheet 17.

FIG. 30 is a flowchart illustrating steps for applying a moldable splint to a patient in the embodiment of the orthotic system shown in FIGS. 24-29.

In the splinting process of FIG. 30, Steps 70, 71 and 73-78 are the same as described above for Steps 50-51, 53-56 and 59-60, respectively, with reference to the splinting process of FIG. 23. Step 72 in FIG. 30 differs from Step 52 in FIG. 23 in that PEF sheet 17 is applied on the injured body part instead of the bandage. Another difference between the splinting processes of FIGS. 23 and 30 is that unlike the option to take off the bandage in Step 57 of FIG. 23, PEF sheet 17 is not removed prior to the end of the splinting process (Step 78). Stated otherwise, PEF sheet 17 is designed to remain in place with the orthosis as part of the orthotic system according to the present disclosure.

The orthotic device according to the present disclosure may be provided in a relatively flat shape or generally in the shape for a specific body part, such as a wrist, ankle, knee or other body part as well as in general sizes, such as large, medium or small. The orthotic device may also be pre-formed in some cases to approximately fit the body part for trial of size or in the case where a more complex structure requires it. The orthotic device can then be heated and custom shaped as described above to specifically fit the body part that it is to support.

Due to its flexibility, healthcare professionals may easily fit the orthosis to the injured body part and adjust the angle by gently allowing the orthosis to conform to the contours of the injured body part. In addition, if necessary, by temporarily and locally heating the material of the orthosis, a required angle for rehabilitation (e.g. spasticity of forearm) may be easily achieved if required to adopt another desired position.

The orthosis according to the present disclosure is easy and fast to use since it is formed of a pre-cut single sheet of material. The orthosis is also reliable to use, due to its anatomical design, with a high level of patient comfort. During the healing process sometimes it is required to change the angle of the injured body part, and this can be achieved with the orthosis of the present disclosure because it can be reshaped a number of times (e.g., more than ten times) to adopt another required position. As such, the orthosis according to the present disclosure is reshapeable and reusable.

Furthermore, the orthosis of the present disclosure is easy and quick to install. The orthosis offers the advantage of being able to be easily and rapidly removed and reapplied to a human or animal body part. The orthosis can therefore be recycled during injury healing. Because the orthosis of the present disclosure is formed of a single sheet of material and not of two or more separate elements which need to be affixed to each other by one or more fastening means, a physiotherapist or physician may rapidly apply and remove the orthosis to/from the injured body part.

The orthosis according to the present disclosure is soft when heated in order to provide optimal wearing comfort to the patient. The orthosis is in particular very light, even three to four times lighter than conventional orthotic devices made of thermoplastic materials other than PLA. Additionally, the orthosis of the present disclosure is clean and free of dust, translucent for X-rays, can be heated and formed in situ onto the patient's body part, can be worn until complete recovery of the injured body part, and has improved resistance to abrasion as compared to conventional orthotic devices. Another advantages of the orthosis according to the present disclosure is that due to its design it lowers both the cost to manufacture and apply the orthosis on the patient's body part, as compared with prefabricated orthotic devices, for example.

Additional advantages are achieved when the orthosis formed of a PLA sheet according to the present disclosure is further used in combination with an inner protective layer formed as a PEF sheet as described above with reference to FIGS. 24-30. For example, due to the thermal insulation property of the PEF sheet, the PLA sheet remains moldable for more than 1 minute, providing more than sufficient time for medical professionals to mold the splint onto the patient's injured body part.

In one embodiment, the orthosis as well as the PLA sheet according to the present disclosure can be fabricated in different colors (e.g., fluorescent colors) and sizes for both children and adults.

In another embodiment, various accessories can be used with the orthosis according to the present disclosure to make each unique. For example, decorative elements may be embedded in the sheet of orthosis material at a depth at which the decorative elements remain joined to the sheet when it is heated and molded into the orthotic shape. Various accessories can also be added to the orthosis (e.g., splint) after it is fully set.

In yet another embodiment, biometric sensors are incorporated onto the orthosis of the present disclosure to permit doctors to monitor various conditions, including temperature, blood flow, swelling sweating, and mechanical pressure applied by the orthosis. Information obtained by the biometric sensors can be transmitted to a hospital/doctor via the internet to allow monitoring of the patient's condition.

The orthosis according to the present disclosure provides a ready to use orthosis solution to healthcare professionals, as compared to conventional orthotic devices comprised of thermoplastic plates that need cutting and trimming in order to form the orthosis. For example, the process for forming and applying an orthosis using thermoplastic plates is both time consuming and cumbersome. It could take anywhere from 1 to 2 hours to form the orthosis because, among other things, the healthcare professional needs to cut the thermoplastic plates, form the plates into the patient's injured part, and trim all of the edges of the resulting orthosis, all before the procedure of applying the orthosis to the patient's injured part is started. In contrast, due to its unique construction, the orthosis according to the present disclosure can substantially reduce the time it takes to apply the orthosis on to the patient's injured body part. The ready-to-use nature of the orthosis according to the present disclosure substantially reduces the average application time required by healthcare professionals. Furthermore, unlike many comparable orthosis devices, the orthosis of the present disclosure is also reusable.

Moreover, the orthosis according to the present disclosure is ready for use anytime needed. The orthosis is not custom, so it can fit any patient. This feature allows doctors and nurses to spend their time more efficiently. Thus, the orthosis according to the present disclosure allows healthcare professionals to reduce their costs and limit their inefficiencies, whilst patients are provided with an excellent quality orthotic device.

The previous description of the disclosure is provided to enable any person skilled in the art to make or use the disclosure. Various modifications to the disclosure will be readily apparent to those skilled in the art, and the generic principles defined herein may be applied to other variations without departing from the scope of the disclosure. Thus, the disclosure is not intended to be limited to the examples and designs described herein but are to be accorded the widest scope consistent with the principles and novel features disclosed herein.

What is claimed is:

1. An orthotic device comprising: a single sheet of moldable Polylactic Acid (PLA) material for embracing and supporting a body part of a human or animal comprising an articulation and configured to maintain the body part in an optimal position for development and treatment.

2. The orthotic device of claim 1, wherein the PLA sheet is formed with a plurality of perforations uniformly distributed over the total surface area of the PLA sheet.

3. The orthotic device of claim 2, wherein the perforations are distributed over 75% to 95% of the total surface area of the PLA sheet.

4. The orthotic device of claim 2, wherein the PLA sheet has a thickness in the range of about 1.5 mm to about 5.0 mm.

5. The orthotic device of claim 2, wherein the PLA sheet has a thickness in the range of about 1.5 mm to about 3.5 mm.

6. The orthotic device of claim 2, wherein the PLA sheet has a thickness in the range of about 1.8 mm to about 2.0 mm.

7. The orthotic device of claim 2, wherein the PLA sheet formed with at least one aperture for passage therethrough of a body part of the human or animal.

8. The orthotic device of claim 1, wherein the PLA sheet is pre-cut to a preselected configuration suitable for forming to shape for a custom application.

9. The orthotic device of claim 1, wherein the PLA sheet is 3D printed.

10. An orthotic system comprising:
a single sheet of moldable Polylactic Acid (PLA) material for embracing and supporting a body part of a human or animal comprising an articulation and configured to maintain the body part in an optimal position for development and treatment; and
a protective layer configured for placement between the PLA sheet and the body part.

11. The orthotic system of claim 10, wherein the protective layer comprises a bandage made of a fabric material.

12. The orthotic system of claim 10, wherein the protective layer comprises a single moldable sheet of polyethylene foam (PEF).

13. The orthotic system of claim 12, wherein the PEF sheet has a thickness in the range of about 1.5 mm to about 5.0 mm.

14. The orthotic system of claim 12, wherein the PEF sheet has a thickness of about 3.0 mm.

15. The orthotic system of claim 10, wherein the PLA sheet and the PEF sheet have substantially the same structural configuration.

16. The orthotic system of claim 10, further comprising a fastener for releasably securing the PLA sheet and the PEF sheet onto the body part.

17. A method of applying a splint to a patient's body to immobilize a body part of the patient's body, comprising the steps of:
providing a pre-cut splint formed of a single sheet of moldable Polylactic Acid (PLA) material;
heating the splint above the glass transition temperature thereof to place the splint in a temporarily elastic state so that it can be molded into the shape of the body part of the patient's body; and
molding the splint in the heated state onto the body part of the patient's body.

18. The method of claim 17, further comprising heating the splint during the molding step.

19. The method of claim 17, further comprising the step of applying a protective layer onto the body part of the patient's body prior to molding the splint, and molding the splint in the heated state onto the applied protective layer.

20. The method of claim 17, wherein the protective layer comprises a single moldable sheet of polyethylene foam (PEP).
